(19) United States
(12) Patent Application Publication
(10) Pub. No.: US 2025/0155906 A1
(43) Pub. Date: May 15, 2025

(54) LIQUID TREATMENT DEVICE AND METHOD OF TREATING A LIQUID

(71) Applicants: BUERKERT WERKE GMBH & CO. KG., Ingelfingen (DE); BURKERT AUSTRALIA PTY LTD, Norwest (AU)

(72) Inventor: Christopher HOEY, Norwest (AU)

(21) Appl. No.: 18/936,287

(22) Filed: Nov. 4, 2024

(30) Foreign Application Priority Data
Nov. 9, 2023 (DE) 102023131167.0

(51) Int. Cl. G05D 23/19; C12Q 3/00

(52) U.S. Cl. CPC G05D 23/1919; C12Q 3/00

(57) ABSTRACT

A liquid treatment device including a tank for receiving and further processing liquid by means of biological and/or chemical processes taking place in the liquid, a tempering jacket which surrounds the tank and is arranged in a fluid circuit in which a tempering fluid circulates, and via which the liquid in the tank is tempered to control biological processes in the liquid, a control valve which is located in the fluid circuit and via which the liquid flow in the tempering jacket can be closed-loop controlled to adjust the temperature of the liquid in the tank, a first temperature sensor which measures the actual temperature of the liquid received in the tank, a second temperature sensor which measures the actual temperature of the tempering fluid present in the tempering jacket, and an electronic closed-loop control unit for the closed-loop control of a temperature of the tempering fluid present in the tempering jacket. The closed-loop control unit includes a primary loop of closed-loop controlling and a secondary loop of closed-loop controlling, a first electronic closed-loop controller being included in the primary loop of closed-loop controlling and a second electronic closed-loop controller being included in the secondary loop of closed-loop controlling.

### TECHNICAL FIELD

The present disclosure relates to a liquid treatment device and to a method of treating a liquid in a liquid treatment device.

### BACKGROUND

Liquid treatment devices within the meaning of the present disclosure serve to further process liquids in a tank by means of biological and/or chemical processes taking place in the liquid, i.e. the liquid is also to be understood to include cells, yeasts or, more generally, biological material, etc., present in the liquid, which are to be specifically treated. The tank is, for example, a tank having a relatively large volume for mixing and/or blending or a storage tank. For example, the tank has a volume of 5 m³ or more.

The temperature of the liquid must be within a defined range such that the biological processes run efficiently. For this purpose, a fluid circuit is usually present in which a tempering fluid circulates, which tempers the liquid received in the tank, in particular by means of a tempering jacket surrounding the tank.

In known liquid treatment devices, tempering fluid flows for example continuously through the fluid circuit. As a result, the energy consumption of the liquid treatment devices is very high. Since the system is sluggish due to the large volume of the tank and a temperature change of the liquid occurs only slowly, it is only possible to react to a change in temperature with a corresponding delay. Consequently, in known liquid treatment devices, an excessive tempering of the liquid regularly occurs, which may cause, for example, lines, valves and/or some of the liquid to freeze. Examples of such liquid treatment devices are bioreactors in which, for example, cell cultures are cultivated, or tanks in which wine and sparkling wine, other alcoholic beverages or even milk are kept at a temperature which is as exact as possible to bring the liquid into a desired state (e.g. by fermentation) or to keep it in a state. Such tanks may contain several thousand liters of liquid. The tempering of the tank is correspondingly energy-intensive and the system is correspondingly sluggish.

It is therefore an object of the present disclosure to specify a liquid treatment device and a method of treating a liquid, by means of which the liquid can be cooled particularly efficiently.

### SUMMARY

Example embodiments disclose a liquid treatment device comprising a tank for receiving and further processing liquid by means of biological processes taking place in the liquid, a tempering jacket which surrounds the tank and is arranged in a fluid circuit in which a tempering fluid circulates, and via which the liquid in the tank is tempered to control biological processes in the liquid, a control valve which is located in the fluid circuit and via which the liquid flow in the tempering jacket can be closed-loop controlled to adjust the temperature of the liquid in the tank, a first temperature sensor which measures the actual temperature of the liquid received in the tank, and a second temperature sensor which measures the actual temperature of the tempering fluid present in the tempering jacket. The liquid treatment device also comprises an electronic closed-loop control unit for the closed-loop control of a temperature of the tempering fluid present in the tempering jacket, the closed-loop control unit comprising a primary loop of closed-loop controlling and a secondary loop of closed-loop controlling. A first electronic closed-loop controller is included in the primary loop of closed-loop controlling and a second electronic closed-loop controller is included in the secondary loop of closed-loop controlling. The closed-loop control unit is configured such that the reference variable of the first closed-loop controller is the desired temperature of the liquid contained in the tank and the actual temperature of the liquid is fed into the first closed-loop controller as a closed-loop control variable, and that the controlled variable of the first closed-loop controller is a desired value for a difference between the temperature of the liquid received in the tank and the temperature of the tempering fluid in the tempering jacket which is necessary to achieve the desired temperature. The desired temperature and the actual temperature of the liquid are in particular indirectly fed into the first closed-loop controller. The actual temperature of the liquid is additionally offset against the controlled variable of the first closed-loop controller to form a desired value of the temperature for the tempering fluid contained in the tempering jacket. The desired value for the temperature of the tempering fluid contained in the tempering jacket forms the reference variable for the second closed-loop controller and the actual temperature of the tempering fluid contained in the tempering jacket is fed as a closed-loop control variable into the second closed-loop controller, the second closed-loop controller determining a controlled variable by means of which it drives the control valve. In particular, the desired value for the temperature of the tempering fluid contained in the tempering jacket and the actual temperature of the tempering fluid contained in the tempering jacket are indirectly fed into the second closed-loop controller.

In particular, the primary loop of closed-loop controlling and the secondary loop of closed-loop controlling form a cascade closed-loop control. As a result, the closed-loop control path is subdivided into smaller sections which are easier to control in a closed loop. In particular, the secondary loop of closed-loop controlling is faster than the primary loop of closed-loop controlling. In other words, the time constant of the secondary loop of closed-loop controlling is smaller than the time constant of the primary loop of closed-loop controlling.

A liquid treatment device according to the present disclosure has the advantage that a temperature of the liquid contained in the tank may be closed-loop controlled particularly precisely. In addition, the closed-loop control is very efficient, so that energy consumption for tempering the liquid is reduced. In particular, excessive tempering of the liquid is avoided when using the liquid treatment device according to the present disclosure.

The design, according to the present disclosure, of the closed-loop control unit for the closed-loop control of the temperature of the tempering fluid also prevents parts of the fluid circuit, in particular the control valve, from freezing. This is due to the fact that the tempering fluid in the tempering jacket is tempered as required and that a temperature difference between the actual temperature of the liquid and the actual temperature of the tempering fluid in the tempering jacket is no greater than is necessary to cool the liquid. The control valve can thus be designed to be smaller than is the case with known solutions.

The temperature sensors measure the temperature at the outlet of the tank or at the outlet of the tempering jacket, for example. This prevents freshly supplied liquid from distorting the measurement result.

The control valve may be an on-off valve or a proportional valve.

A feed pump, for example, is arranged in the fluid circuit so that a sufficiently strong circulation of the tempering fluid is ensured.

The controlled variable of the second closed-loop controller is preferably a switching signal for switching the control valve. Thus, based on the controlled variable of the second closed-loop controller, a volume flow of the tempering fluid can be set in the fluid circuit by driving the control valve in accordance with the controlled variable of the second closed-loop controller. In this way, the desired temperature is set in the tempering jacket.

The second closed-loop controller is designed, for example, to output a pulse-width modulated switching signal. Compared to the known liquid treatment devices, in which the control valve is sometimes open for several hours, the control valve is thus only open for a short time, which has a positive effect on energy consumption, which is thus significantly reduced. The temperature of the tempering fluid is subject to less severe temperature fluctuations with such a closed-loop control.

The period of the pulse-width modulated switching signal is preferably between 100 seconds and 600 seconds, in particular 300 seconds. The period is particularly adapted to the inertia of the system. This means that excessive energy input within a short time is avoided, as a result of which the liquid in the tank can be tempered particularly evenly and continuously.

For example, the on-time of the pulse-width modulated switching signal is between 1 second and 10 seconds, in particular 3 seconds. This contributes to prevent the control valve and the lines of the fluid circuit from freezing.

The first closed-loop controller and/or the second closed-loop controller is/are, for example, a PID controller. PID controllers are particularly fast and stable, as a result of which a desired value can be set particularly accurately and maintained permanently. In particular, a PID controller can be used to react particularly quickly to disturbances and make an appropriate adjustments in the closed-loop control.

According to one embodiment, a first fluid circuit and a second fluid circuit are present, the tempering jacket being included in both fluid circuits, and the two fluid circuits each carrying a differently tempered tempering fluid, the first fluid circuit carrying in particular cold water and the second fluid circuit carrying hot water. Thus, the temperature of the tempering fluid in the tempering jacket can be both actively increased and decreased.

The two circuits preferably use the same inlet to the tempering jacket, which allows a particularly compact design of the liquid treatment device. In this case, preferably four valves are present which closed-loop control a fluid flow, namely two valves at the inlet and two valves at the outlet of the tempering jacket. This prevents hot and cold water from mixing.

For example, a light sensor, an ambient temperature sensor, an energy cost indicator and/or a time-recording means are provided, the brightness, the ambient temperature, the energy costs and the time of day and/or year being fed into the closed-loop control unit, the closed-loop control unit being set up to closed-loop control a temperature of the tempering fluid present in the tempering jacket based on the aforementioned parameters when the energy costs are below the average energy costs for the day. For example, a period in which the tempering is carried out depends on the own-electricity generation by means of wind and/or photovoltaics or on the availability of night current. In this way, the energy efficiency of the liquid treatment device is further improved and costs can be reduced.

The liquid treatment device has, for example, a display for showing a current or an average energy consumption.

For example, the closed-loop control unit comprises a first electronic comparison unit, which is coupled in terms of signaling to the first closed-loop controller and is designed to subtract the desired temperature of the liquid contained in the tank and the actual temperature of the liquid in the tank from each other and to transfer the closed-loop control difference to the first closed-loop controller. The closed-loop control difference is, in particular, the difference between the desired temperature of the liquid contained in the tank and the actual temperature of the liquid in the tank. The first closed-loop controller is set up to form a controlled variable according to the closed-loop control difference such that the difference approaches zero. It is thus achieved that the actual temperature of the liquid approaches the desired temperature and that the desired process for further treatment of the liquid contained in the tank can proceed as optimally as possible.

Furthermore, the closed-loop control unit may comprise a second electronic comparison unit which subtracts the controlled variable of the first closed-loop controller and the actual temperature of the liquid from each other and forms a difference value. Specifically, the desired value for the temperature of the tempering fluid contained in the tempering jacket is calculated in the comparison unit by subtracting the controlled variable of the first closed-loop controller, i.e. the difference between the temperature of the liquid received in the tank and the temperature of the tempering fluid required to achieve the desired temperature, from the actual temperature of the liquid. The reference variable of the second closed-loop controller can thus be calculated by means of the second electronic comparison unit. The efficiency of the liquid treatment device is significantly improved by the fact that the reference variable of the second closed-loop controller is calculated in the closed-loop control unit and is not specified by a user. In particular, the reference variable is variable and continuously adapted to the temperature conditions prevailing in the liquid treatment device.

A third electronic comparison unit is preferably provided, which is coupled in terms of signaling to the second closed-loop controller and is designed to receive the desired value formed by the second comparison unit and to form a difference value with the actual temperature of the tempering fluid, which is transferred to the second closed-loop controller as a closed-loop control difference. Since the difference value formed by the second comparison unit represents a desired temperature of the tempering fluid contained in the tempering jacket, the difference value formed by the third comparison unit corresponds to a difference between the desired temperature and the actual temperature of the tempering fluid contained in the tempering jacket. Consequently, a controlled variable of the second closed-loop controller can be formed such that the difference approaches zero, as a result of which the desired temperature is set in the tempering jacket.

The object is further achieved by a method of treating a liquid in a liquid treatment device, which is in particular configured as described above, comprising a tank for receiving and further processing liquid by means of biological processes taking place in the liquid, a tempering jacket which surrounds the tank and is arranged in a fluid circuit in which a tempering fluid circulates, and via which the liquid in the tank is tempered to control biological processes in the liquid, a control valve which is located in the fluid circuit and via which the liquid flow in the tempering jacket can be closed-loop controlled to adjust the temperature of the liquid in the tank, a first temperature sensor which measures the actual temperature of the liquid received in the tank, a second temperature sensor which measures the actual temperature of the tempering fluid present in the tempering jacket, and an electronic closed-loop control unit for the closed-loop control of a temperature of the tempering fluid present in the tempering jacket, the closed-loop control unit comprising a primary loop of closed-loop controlling and a secondary loop of closed-loop controlling, a first electronic closed-loop controller being included in the primary loop of closed-loop controlling and a second electronic closed-loop controller being included in the secondary loop of closed-loop controlling, and the reference variable of the first closed-loop controller being the desired temperature of the liquid contained in the tank.

In a first step of the method according to the present disclosure, the actual temperature of the liquid is fed directly or indirectly as a closed-loop control variable into the first closed-loop controller, the controlled variable of which is a desired value for a difference between the temperature of the liquid contained in the tank and the temperature of the tempering fluid in the tempering jacket which is necessary to achieve the desired temperature. In a further step, the actual temperature of the liquid is offset against the controlled variable of the first closed-loop controller to form a desired value of the temperature for the tempering fluid contained in the tempering jacket. The desired value for the tempering fluid contained in the tempering jacket forms indirectly or directly the reference variable for the second closed-loop controller. The actual temperature of the tempering fluid contained in the tempering jacket is fed as a closed-loop control variable into the second closed-loop controller, the second closed-loop controller determining a controlled variable by means of which it drives the control valve.

As already described in connection with the liquid treatment device according to the present disclosure, the method allows a temperature of the liquid contained in the tank to be closed-loop controlled particularly precisely.

To form a desired value for the temperature of the tempering fluid contained in the tempering jacket, in particular a difference is formed from the actual temperature of the liquid and the controlled variable of the first closed-loop controller, as already described in connection with the liquid treatment device. The reference variable of the second closed-loop controller is thus not specified by a user, but calculated by the closed-loop control unit.

### BRIEF DESCRIPTION OF THE DRAWINGS

FIG. 1 schematically shows a liquid treatment device according to the present disclosure, and

FIG. 2 schematically shows a further liquid treatment device according to the present disclosure.

### DETAILED DESCRIPTION

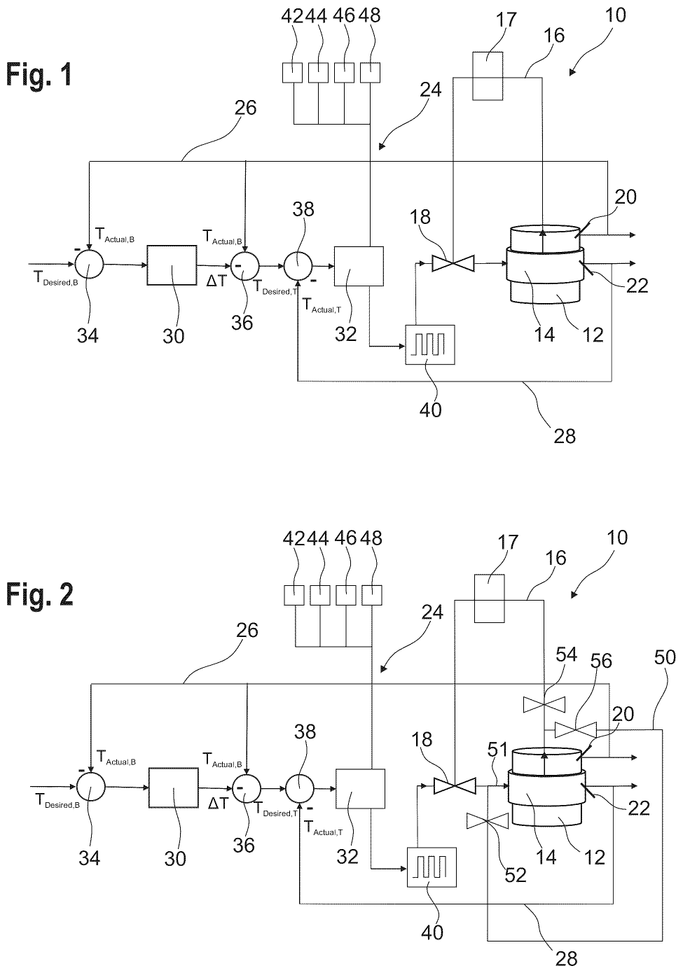

FIG. 1 schematically shows a liquid treatment device 10, for example in wine or sparkling wine cellars, a dairy or a cell culture production plant. In addition to the structural features of the liquid treatment device 10, the following description also illustrates a method of treating a liquid in the liquid treatment device 10.

The liquid treatment device 10 comprises a tank 12 for receiving and further processing liquid by means of biological processes taking place in the liquid. For example, the tank contains wine, milk products or microorganisms to be cultured, cell cultures, etc.

The liquid treatment device 10 is particularly designed to cool or heat the liquid contained in the tank 12, i.e. to bring it to the temperature at which the biological process runs optimally and is then possibly also delayed or stopped when the desired product has been achieved.

For this purpose, the tank 12 is surrounded by a tempering jacket 14.

The tank 12 is, for example, a stainless steel tank having a capacity of several hundred or several thousand liters.

The tempering jacket 14 is, for example, a jacket that is guided separately around the outside of the tank or is integrated into the outer wall of the tank 12.

The tempering jacket 14 is arranged in a fluid circuit 16 in which a tempering fluid circulates. The tempering fluid is, for example, water, in particular salt water, or glycol.

The fluid circuit 16 contains, for example, a storage tank 17 for the tempering fluid, in which tempering fluid is stored at a defined temperature.

The liquid in the tank 12 can be tempered via the tempering jacket 14 to control biological processes in the liquid. Specifically, the liquid in the tank 12 is tempered such that the desired processes can take place optimally.

The liquid treatment device 10 also comprises a control valve 18, which is located in the fluid circuit 16 and via which the liquid flow in the tempering jacket 14 can be closed-loop controlled to adjust the temperature of the liquid in the tank 12.

In addition, a first temperature sensor 20, which measures the actual temperature of the liquid received in the tank 12, and a second temperature sensor 22, which measures the actual temperature of the tempering fluid present in the tempering jacket 14, are provided.

The temperature of the tempering fluid present in the tempering jacket 14 is closed-loop controlled by means of an electronic closed-loop control unit 24, which comprises a primary loop of closed-loop controlling 26 and a secondary loop of closed-loop controlling 28, the two loops of closed-loop controlling 26, 28 forming in particular a cascade closed-loop control.

The secondary loop of closed-loop controlling 28 is in particular included in the primary loop of closed-loop controlling 26 and has a higher processing speed than the primary loop of closed-loop controlling 26.

The primary loop of closed-loop controlling 26 includes a first electronic closed-loop controller 30 and the secondary loop of closed-loop controlling 28 includes a second electronic closed-loop controller 32.

Both the first closed-loop controller 30 and the second closed-loop controller 32 are preferably PID controllers.

Furthermore, the closed-loop control unit 24 includes electronic comparison units 34, 36, 38 which are designed to offset values against each other, which will be described in detail below. The first and the second electronic comparison unit 34, 36 are connected in terms of signaling to the first closed-loop controller 30, the first electronic comparison unit 34 being located at the input of the first closed-loop controller 30 and the second electronic comparison unit 34 being located at the output of the first closed-loop controller 30. The third electronic comparison unit 38 is connected in terms of signaling to the second closed-loop controller 32 and is in particular arranged at the input of the second closed-loop controller 32, i.e. between the second comparison unit 36 and the second closed-loop controller 32.

The closed-loop control unit 24 is configured such that the reference variable of the first closed-loop controller 30 is indirectly the desired temperature T_{Desired,B} of the liquid contained in the tank 12.

The actual temperature T_{Actual,B} of the liquid is fed into the first closed-loop controller 30 as a closed-loop control variable.

Specifically, the desired temperature T_{Desired,B} and the actual temperature T_{Actual,B} are indirectly fed into the first closed-loop controller 30, because the comparison unit 34 is connected upstream.

More precisely, the difference value between the desired temperature T_{Desired,B} and the actual temperature T_{Actual,B} of the liquid in the tank is formed in the first electronic comparison unit 34, which is coupled in terms of signaling to the first closed-loop controller 30 and is configured to subtract the desired temperature T_{Desired,B} of the liquid contained in the tank and the actual temperature T_{Actual,B} of the liquid from each other, and the difference value is transferred as a closed-loop control variable to the first closed-loop controller 30.

The first closed-loop controller 30 is configured such that the controlled variable of the first closed-loop controller 30 is a desired value for a difference ΔT between the actual temperature T_{Actual,B} of the liquid received in the tank 12 and the temperature of the tempering fluid in the tempering jacket 14 which is necessary to achieve the desired temperature T_{Desired,B}, i.e. the desired temperature T_{Desired,T} of the tempering fluid.

The difference ΔT is further offset against the actual temperature T_{Actual,B} of the liquid, in particular in the second electronic comparison unit 36, to form the desired value T_{Desired,T} of the temperature for the tempering fluid contained in the tempering jacket 14.

More precisely, the second electronic comparison unit 36 subtracts the controlled variable of the first closed-loop controller, i.e. the difference ΔT, and the actual temperature T_{Actual,B} of the liquid from each other and forms a difference value. The difference value thus obtained corresponds to the desired temperature T_{Desired,T} of the tempering fluid.

In other words, the following calculation takes place in the second electronic comparison unit 36:

T_{Desired,T}=T_{Actual,B}−ΔT, where ΔT=T_{Actual,B}-T_{Desired,T}.

The desired value T_{Desired,T} for the tempering fluid contained in the tempering jacket 14 forms the indirect reference variable for the second closed-loop controller 32 and the actual temperature T_{actual,T} of the tempering fluid contained in the tempering jacket 14 is fed into the second closed-loop controller 32 as a closed-loop control variable, the second closed-loop controller 32 determining a controlled variable by means of which it drives the control valve 18.

More precisely, the desired value T_{Desired,T} calculated in this way is transferred to the third electronic comparison unit 38.

The third electronic comparison unit 38 is configured to receive the difference value T_{Desired,T} formed by the second comparison unit 36 and to form a further difference value with the actual temperature T_{actual,T} of the tempering fluid, which is transferred to the second closed-loop controller 32 as a closed-loop control variable.

Based on the closed-loop control variable, the second closed-loop controller 32 closed-loop controls the temperature of the tempering fluid in the tempering jacket 14.

Specifically, the controlled variable of the second closed-loop controller 32 is a switching signal 40 for switching the control valve 18, which is schematically illustrated in FIG. 1.

The switching signal 40 controls the opening and closing times of the control valve 18, as a result of which a fluid flow of tempering fluid flowing into the tempering jacket 14 is regulated. Thus, the temperature of the tempering fluid in the tempering jacket 14 is regulated.

For example, the switching signal 40 is a pneumatic signal, in particular when the control valve 18 has a pneumatic valve drive. However, the switching signal can also be an electronic signal.

More precisely, the second closed-loop controller 32 is configured to output a pulse-width modulated switching signal which forms the switching signal 40. As a result, the control valve 18 is only open for a short time, which has a favorable effect on energy consumption.

The period of the pulse-width modulated switching signal is, for example, between 100 seconds and 600 seconds, in particular 300 seconds.

The on-time of the pulse-width modulated switching signal is, for example, between 1 second and 10 seconds, in particular 3 seconds.

Optionally, the liquid treatment device 10 can include a light sensor 42, an ambient temperature sensor 44, an energy cost indicator 46 and/or a time-recording means 48, which are shown schematically in FIG. 1.

In such a liquid treatment device 10, the brightness, the ambient temperature, the energy costs and the time of day and/or year can be fed into the closed-loop control unit 24, the closed-loop control unit 24 being set up to closed-loop control a temperature of the tempering fluid present in the tempering jacket 14 based on the aforementioned parameters when the energy costs are below the average energy costs for the day, in particular when the energy costs are the lowest. The light sensor 42, for example, makes it possible to determine whether there is enough daylight to produce electricity using a photovoltaic system. The time-recording means 48 can be used, for example, to determine whether cheaper night-time electricity is available. The energy cost indicator 46 provides for example information as to the amount of the current energy costs.

The aforementioned parameters, for example, are fed into the second closed-loop controller 32.

A display may be present on which a user can see the consumption data, the available energy, the cycle times of the control valve 18 and/or the temperature of the liquid contained in the tank and the temperature of the tempering fluid contained in the tempering jacket 14.

The second closed-loop controller 32 is set up, for example, to output a switching signal only if the energy costs are below a threshold value and/or if the temperature difference between the desired value T_{Desired,T} of the tempering fluid contained in the tempering jacket 14 and the actual value T_{actual,T} of the tempering fluid contained in the tempering jacket 14, which is output by the third comparison unit 38, exceeds a threshold value.

FIG. 2 schematically illustrates a further embodiment of the liquid treatment device 10.

In the liquid treatment device 10 according to FIG. 2, a first fluid circuit 16 and a second fluid circuit 50 are provided, the tempering jacket 14 being contained in both fluid circuits 16, 50.

The two fluid circuits 16, 50 have a common inlet 51 to the tempering jacket 14.

The two fluid circuits 16, 50 can each contain a differently tempered tempering fluid.

For example, the first fluid circuit 16 can carry cold water and the second fluid circuit 50 can carry hot water.

In such an embodiment, there are preferably four valves, in particular two control valves 18, 52 which each separately closed-loop control the inflow of the differently tempered tempering fluids at different temperatures, and two valves 54, 56 which control the return of the tempering fluids into the corresponding fluid circuit 16, 50 at the outlet of the tempering jacket 14.

While the disclosure has been described with reference to a preferred embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A liquid treatment device, comprising:
a tank for receiving and further processing liquid through biological and/or chemical processes taking place in the liquid,
a tempering jacket which surrounds the tank and is arranged in a fluid circuit in which a tempering fluid circulates, and via which the liquid in the tank is tempered to control biological processes in the liquid,
a control valve which is located in the fluid circuit and via which the liquid flow in the tempering jacket can be closed-loop controlled to adjust the temperature of the liquid in the tank,
a first temperature sensor which measures the actual temperature of the liquid received in the tank,
a second temperature sensor which measures the actual temperature of the tempering fluid present in the tempering jacket, and
an electronic closed-loop control unit for the closed-loop control of a temperature of the tempering fluid present in the tempering jacket,
wherein the closed-loop control unit includes a primary loop of closed-loop controlling and a secondary loop of closed-loop controlling,
wherein a first electronic closed-loop controller is included in the primary loop of closed-loop controlling and a second electronic closed-loop controller being included in the secondary loop of closed-loop controlling,
wherein the closed-loop control unit is configured such that the reference variable of the first closed-loop controller is the desired temperature of the liquid contained in the tank and the actual temperature of the liquid is fed into the first closed-loop controller as a closed-loop control variable, and that the controlled variable of the first closed-loop controller is a desired value for a difference between the temperature of the liquid received in the tank and the temperature of the tempering fluid in the tempering jacket which is necessary to achieve the desired temperature, and the actual temperature of the liquid is additionally offset against the controlled variable of the first closed-loop controller to form a desired value of the temperature for the tempering fluid contained in the tempering jacket, and
wherein the desired value for the temperature of the tempering fluid contained in the tempering jacket forms the reference variable for the second closed-loop controller and the actual temperature of the tempering fluid contained in the tempering jacket is fed as a closed-loop control variable into the second closed-loop controller, the second closed-loop controller determining a controlled variable by means of which it drives the control valve.

2. The liquid treatment device according to claim 1, wherein the controlled variable of the second closed-loop controller is a switching signal for switching the control valve.

3. The liquid treatment device according to claim 2, wherein the second closed-loop controller is configured to output a pulse-width modulated switching signal.

4. The liquid treatment device according to claim 3, wherein the period of the pulse-width modulated switching signal is between 100 seconds and 600 seconds.

5. The liquid treatment device according to claim 3, wherein the on-time of the pulse-width modulated switching signal is between 1 second and 10 seconds.

6. The liquid treatment device according to claim 1, wherein the first closed-loop controller and/or the second closed-loop controller is/are a PID controller.

7. The liquid treatment device according to claim 1, wherein a first fluid circuit and a second fluid circuit are provided, the tempering jacket being contained in both fluid circuits and the two fluid circuits each carrying a differently tempered tempering fluid.

8. The liquid treatment device according to claim 7, wherein the first fluid circuit carrying cold water and the second fluid circuit carrying hot water.

9. The liquid treatment device according to claim 1, wherein a light sensor, an ambient temperature sensor, an energy cost indicator and/or a time-recording devices are provided, the brightness, the ambient temperature, the energy costs and the time of day and/or year being fed into the closed-loop control unit, the closed-loop control unit being set up to closed-loop control a temperature of the tempering fluid present in the tempering jacket based on the aforementioned parameters when the energy costs are below the average energy costs for the day.

10. The liquid treatment device according to claim 1, wherein the closed-loop control unit comprises a first electronic comparison unit which is coupled in terms of signaling to the first closed-loop controller and which is configured to subtract the desired temperature of the liquid contained in the tank and the actual temperature of the liquid in the tank from each other, and wherein the closed-loop control difference is transferred to the first closed-loop controller.

11. The liquid treatment device according to claim 1, wherein the closed-loop control unit comprises a second electronic comparison unit which subtracts the controlled variable of the first closed-loop controller and the actual temperature of the liquid from each other and forms a difference value.

12. The liquid treatment device according to claim 11, wherein a third electronic comparison unit is provided, which is coupled in terms of signaling to the second closed-loop controller and is configured so as to receive the desired value formed by the second comparison unit and forms a difference value with the actual temperature of the tempering fluid, which is transferred to the second closed-loop controller as a closed-loop control difference.

13. A method of treating a liquid in a liquid treatment device, comprising a tank for receiving and further processing liquid through biological and/or processes taking place in the liquid, a tempering jacket which surrounds the tank and is arranged in a fluid circuit in which a tempering fluid circulates, and via which the liquid in the tank is tempered to control biological processes in the liquid, a control valve which is located in the fluid circuit and via which the liquid flow in the tempering jacket can be closed-loop controlled to adjust the temperature of the liquid in the tank, a first temperature sensor which measures the actual temperature of the liquid received in the tank, a second temperature sensor which measures the actual temperature of the tempering fluid present in the tempering jacket, and an electronic closed-loop control unit for the closed-loop control of a temperature of the tempering fluid present in the tempering jacket, the closed-loop control unit comprising a primary loop of closed-loop controlling and a secondary loop of closed-loop controlling, a first electronic closed-loop controller being included in the primary loop of closed-loop controlling and a second electronic closed-loop controller being included in the secondary loop of closed-loop controlling, and the reference variable of the first closed-loop controller being the desired temperature of the liquid contained in the tank,
the method comprising:
providing the actual temperature of the liquid as a closed-loop control variable into the first closed-loop controller, which determines a controlled variable in the form of a desired value for a difference between the temperature of the liquid contained in the tank and the temperature of the tempering fluid in the tempering jacket which is necessary to achieve the desired temperature,
offsetting the actual temperature of the liquid against the controlled variable of the first closed-loop controller to form a desired value of the temperature for the tempering fluid contained in the tempering jacket, and
providing the desired value for the tempering fluid contained in the tempering jacket to form the reference variable for the second closed-loop controller and the actual temperature of the tempering fluid contained in the tempering jacket is fed as a closed-loop control variable into the second closed-loop controller, the second closed-loop controller determining a controlled variable by means of which it drives the control valve.

14. The method according to claim 13, wherein a difference between the actual temperature of the liquid and the controlled variable of the first closed-loop controller is formed in an electronic comparison unit to form a desired value for the temperature of the tempering fluid contained in the tempering jacket.
